(19) United States
(12) Patent Application Publication
(10) Pub. No.: US 2005/0203585 A1
(43) Pub. Date: Sep. 15, 2005

(54) Water electrode

(75) Inventor: Wade E. Hairfield SR., Las Vegas, NV (US)

Correspondence Address: APRIL 2000 LLC, 3225 McLEOD DRIVE, #100, LAS VEGAS, NV, 89121, US

(73) Assignee: Best Health Products, Inc.

(21) Appl. No.: 10/782,638

(22) Filed: Feb. 19, 2004

(51) Int. Cl. A61N 1/18

(52) U.S. Cl. 607/46

(57) ABSTRACT

An electrode for a foot bath allows a small current to create a tingle in your feet. The AC/DC power supply and stainless steel electrode is portable for use in any plastic tub or bowl. A stand allows use in metal bowl.

### FIELD OF INVENTION

The present invention relates to an electrode that creates a trickle current in a bucket of water that is sized to accommodate a person's feet so as to induce a mild current flow through the person's feet.

### BACKGROUND OF THE INVENTION

The human body is made of about 80% water and further comprises salts as well as complex current generators. Science is far from understanding the complex inter-relationships between the human body and its immediate electrical environment. The art of acupuncture dates back centuries, and yet its theories of operation are still not understood.

It is known that too much electricity passing through a human, can destroy tissue and/or kill the human. Modern physical therapy devices do use surface electrical charges along muscles to promote the strengthening and healing of the muscles. A brief summary of related art follows below.

U.S. Pat. No. 3,198,726 (1965) to Trikilis discloses an ionizer to create de-odorizing heavy oxygen molecules in air.

U.S. Pat. No. 4,057,053 (1977) to Kunz discloses a foot bath massager with a heater and a vibrator.

U.S. Pat. No. 4,429,687 (1984) to Friedson et al. discloses a foot heater and massager.

U.S. Pat. No. 4,435,266 (1984) to Johnston discloses an anode and a cathode in water bath functioning as an electroplating device.

U.S. Pat. No. 4,461,744 (1984) to Erni et al. discloses an ozone generator.

U.S. Pat. No. 4,513,735 (1985) to Friedson et al. discloses a foot massager/heater.

U.S. Pat. No. 4,807,602 (1989) to Scarborough et al. discloses a foot massager/heater.

U.S. Pat. No. 5,098,415 (1992) to Levin discloses a foot bath with an ozone generator.

U.S. Pat. No. 5,367,720 (1994) to Stephens et al. discloses a vertical bath for a foot.

U.S. Pat. No. 5,665,141 (1997) to Vago discloses an ultrasonic bath for treating wounds.

U.S. Pat. No. 5,834,031 (1998) to Martin et al. discloses an ozone bath for treating infected feet.

U.S. Pat. No. 6,309,366 (2001) to Maxwell discloses a foot massager using water jets.

U.S. Pat. No. 6,438,768 (2002) to Yen discloses a foot massager using water jets.

U.S. Pat. No. 6,598,244 (2003) to Yeh discloses a foot massager with ozone generator.

The present invention provides a foot bath using water with a quantity of impurities therein, such that an electrode having an anode and a cathode passes a trickle current through the water. The human's feet are placed adjacent the electrode, thereby inducing a small trickle current through the human's feet. Electrical safeguards are employed to ensure that no harmful level of electricity passes through the human's feet.

Possibly pleasing tingling sensations may be felt by the human, and unknown health benefits may be provided to the human by using the electrical foot bath.

### SUMMARY OF THE INVENTION

An aspect of the present invention is to provide a safe, low level, trickle charge to a foot bath.

Other aspects of this invention will appear from the following description and appended claims, reference being made to the accompanying drawings forming a part of this specification wherein like reference characters designate corresponding parts in the several views.

The present invention has an AC/DC power supply with built in safety circuits to limit the current across an anode and a cathode to a range of about 0-4 amps. The anode/cathode electrode assembly preferably consists of an outer pipe segment about 76.2 mm (three inches) high which surrounds an inner pipe segment about 101.6 mm (four inches) high. Each pipe segment is electrically insulated from each other, wherein they become an anode/cathode when connected to the AC/DC power supply. The ammeter displays the current flow when the electrode is immersed in a foot bath. If the water is very pure, then a small amount of salt is added to create a current flow. The user can set a timer to enjoy his foot bath, and he can vary the current flow to induce a tingling sensation if he wishes.

### BRIEF DESCRIPTION OF THE DRAWINGS

FIG. 1 is a top perspective view of the preferred embodiment electrode with an insulating drainage stand for use with a metal tub.

FIG. 2 is a side elevation view of the electrode in FIG. 1.

FIG. 3 is a top plan view of the electrode in FIG. 1.

FIG. 4 is a bottom plan view of the electrode in FIG. 1.

FIG. 5 is an exploded view of one insulated connector bolt.

FIG. 6 is a top perspective view of an alternate embodiment electrode.

FIG. 7 is a side perspective view of the preferred embodiment in use.

FIG. 8 is a front elevation view of the AC/DC power supply.

FIG. 9 is a top perspective view of the electrode in use with arrows showing areas of current flow.

FIG. 10 is a schematic view of the electrostatic fields generated by the electrode in a conductive fluid such as salt water.

FIG. 11 is a side elevation view of the insulating drainage stand in FIG. 1.

FIG. 12 is a top plan view of the insulating drainage stand in FIG. 1.

FIG. 13 is a bottom plan view of the insulating drainage stand in FIG. 1.

FIG. 14 is a schematic diagram of the AC/DC power supply connected to the electrode.

FIG. 15 is a graph showing water potential versus distance from the electrode.

Before explaining the disclosed embodiment of the present invention in detail, it is to be understood that the invention is not limited in its application to the details of the particular arrangement shown, since the invention is capable of other embodiments. Also, the terminology used herein is for the purpose of description and not of limitation.

### DETAILED DESCRIPTION OF THE DRAWINGS

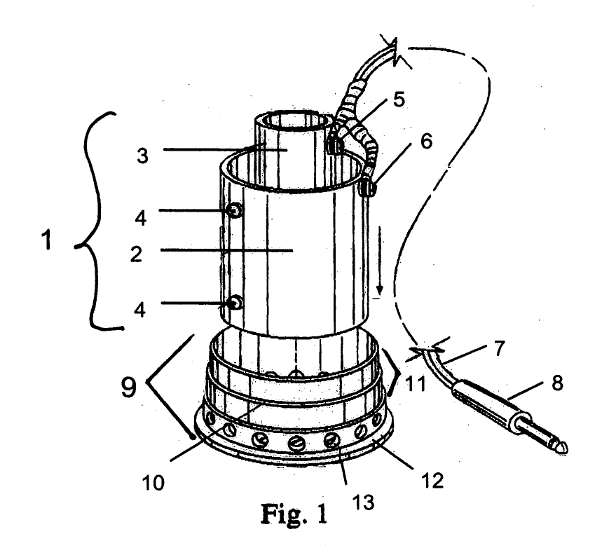

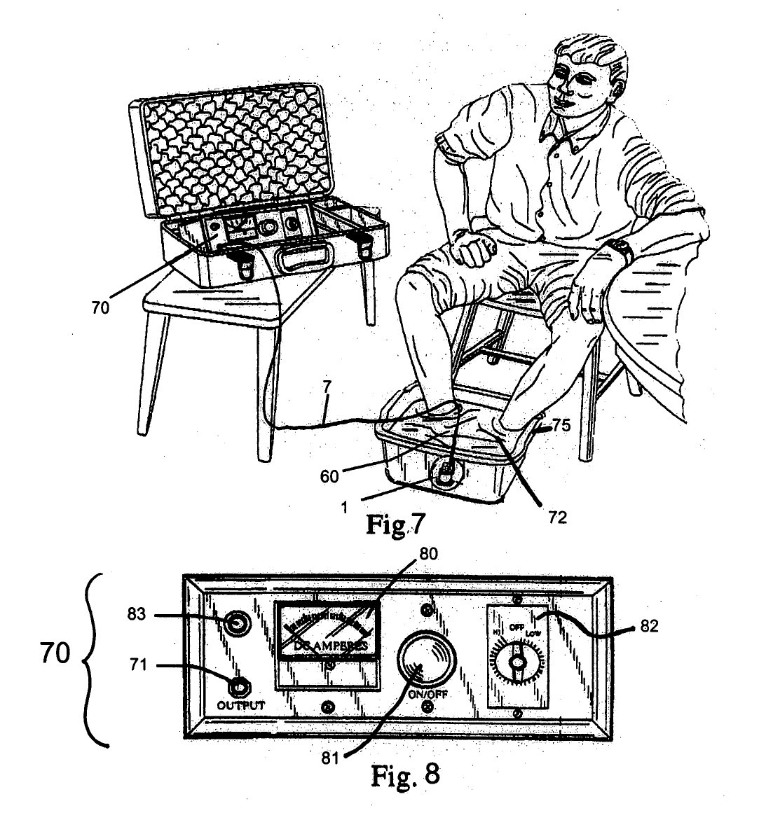

Referring first to FIG. 1 an electrode 1 consists of an anode 3 having a plus power cord connection 5, and cathode 2 having a negative power cord connection 6. The power cord 7 has a connector 8 for insertion into the DC output jack 71 of an AC/DC power supply 70, see FIGS. 7, 8. Insulated bolts 4 support the anode 3 inside the cathode 2. The power connections 5,6 can be reversed.

If the electrode 1 is used in a metal container, then an insulating stand 9 is used to prevent a short circuit between anode 3 and cathode 2. The stand 9 has a bottom 12 and sidewall 11. The sidewall 11 has a ledge 10 to support the cathode 2. Drainage holes 13 permit the foot bath water (FIG. 7, 72) to flow to the cathode 2 and the anode 3.

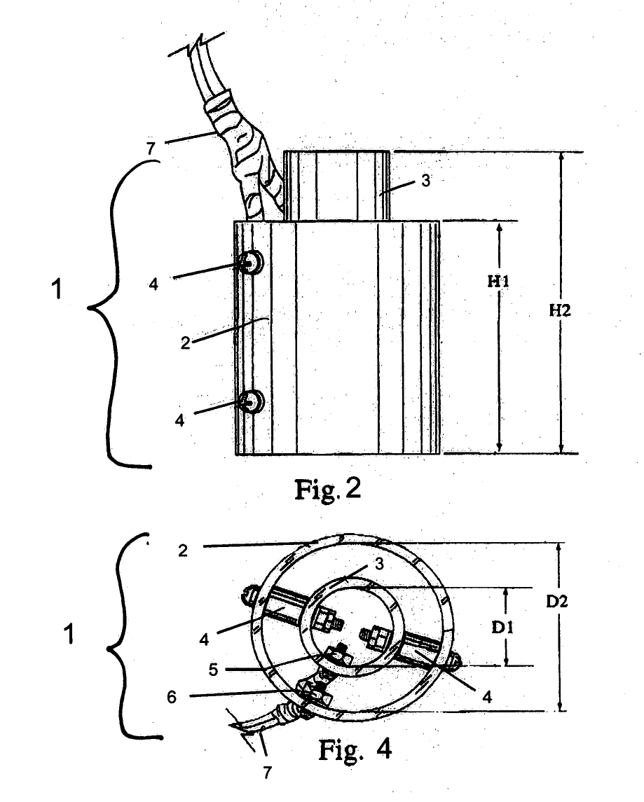

Referring next to FIG. 2-4 nominally the anode 3 is made of a stainless steel pipe segment with a one inch inside diameter, D1=25.4 mm (1″), and a height 42=101.6 mm (four inches}. Cathode 2 is made of a stainless steel pipe segment wherein D2=50.8 mm (2″), and H1=76.2 mm (3″). The relative anode 3 to cathode 2 surface areas are computed as:

$$\frac{( Π {r}^{2} ) ( H1 )}{( Π {r}^{2} ) ( H1 )} = \frac{{Π ( D1 / 2 )}^{2} ( 4 )}{{Π ( D2 / 2 )}^{2} ( 3 )} = \frac{{( 1 / 2 )}^{2} 4}{{( 1 )}^{2} 3} = 1 / 3$$

Therefore, a ratio of anode to cathode of about 1:3 is the best mode, and the wiring could be reversed which would reverse the ratio to 3:1.

Referring next to FIG. 5 the bolt 4 consists of a metal bolt 50, insulated nylon gaskets 51, 53, a nylon sleeve 52, a metal nut 54, and a locking nut 55.

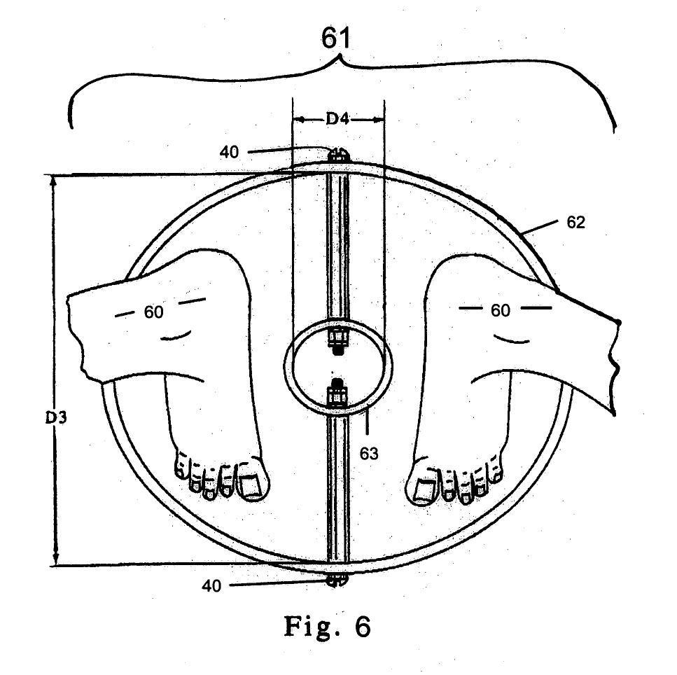

Referring next to FIG. 6 an electrode 61 is large enough to fit the user's feet 60 between the anode 63 and the cathode 62. Bolts 40 hold the anode/cathode together with D3=about 457 mm (18 inches) and D4=about 101.6 mm (4 inches). There may be more current flowing through the feet 60 with this design.

Referring next to FIGS. 7, 8 the electrode 1 is shown in use with the AC/DC power supply 70. The user's feet 60 are in a tub 75 which is preferably made of plastic. Water having impurities 72 is somewhat conductive. The user turns on the AC/DC power supply with switch 81, thereby turning on indicator light 83. He sets the timer 82 to a desired bath time such as ten minutes. He adjusts the amps from 0 to 4 amps to receive whatever sensations he desires.

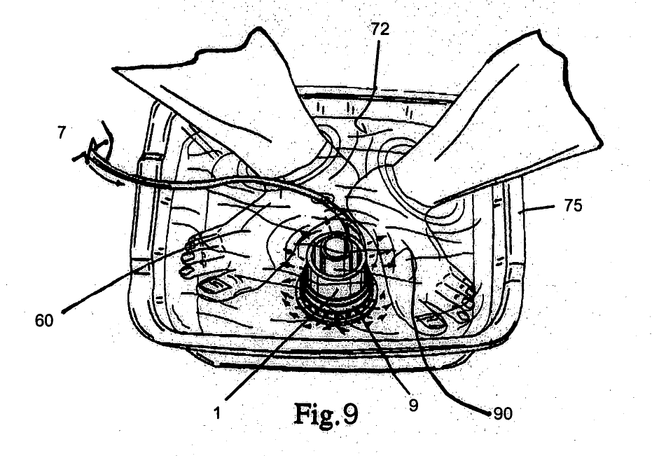

Referring next to FIG. 9 a stand 9 is used in the tub 75. The arrows 90 signify approximate range of electrostatic charges around the electrostatic charges around the electrode 1.

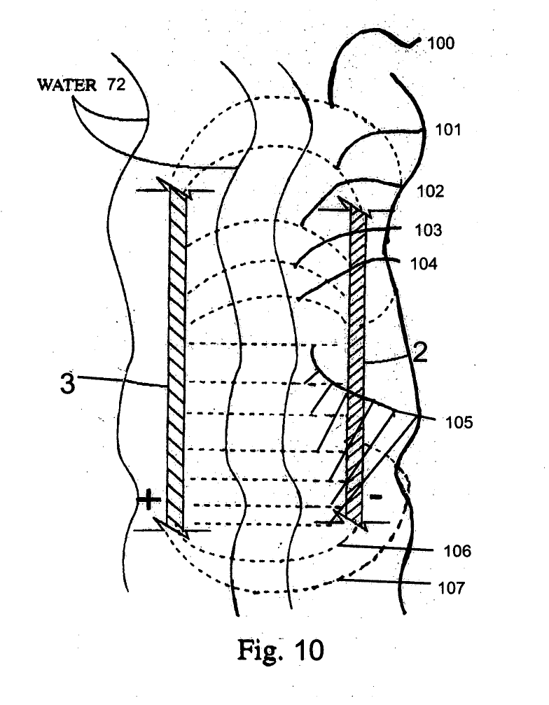

Referring next to FIG. 10 the dotted lines show approximate electrostatic field lines, wherein each line connects the anode 3 to the cathode 3 via the water 72. The fields 105 that run straight between anode and cathode have the strongest currents. The peripheral fields 100-104 and 106-107 are longer and weaker.

### Nominal Instructions for Use

Plug the module cable into the unit (i.e. power supply) marked output.

Place the unit away from the foot bath on a flat dry, stable surface. Plug the power supply into a grounded receptacle in the wall.

Fill the plastic container with just enough warm water to cover the top of the water module. Place the module in the foot bath.

Set the timer for 35 minutes (timer must be on for the machine to work) and then turn the power switch on by turning the knob to the right just a little until it clicks. Note: this is the “On” position.
The green indicator light should come on.

The power read-out should display between 1 and 2 amps, meaning your water conductivity is correct.

If the “Power Read Out” display is less than 1, the water conductivity is too low, add a pinch of salt to the water around the perimeter of the container. Do not sprinkle salt directly over the water module (this will cause unnecessary wear and tear on the module). Stir the water gently with your hand or feet until the salt dissolves. The “Power Read Out” should now be between 1 and 2 amps. To decrease amps, turn Power Control Knob to the left. To increase amps, turn Power Control Knob to the, right.

If the “Power Read Out” display is more than 2, the water conductivity is too high. Turn the Power Control Knob to the left to decrease the amps.

If your readout shows less than 1 or more than 2, refer to the Water Bio Electric section. If your circuit breaker trips, then turn the power control knob to reduce power, and try again.

Prior to a foot bath, remove all jewelry/metal in your body (Including toe rings and ankle bracelets).

Immediately before a foot bath session, you may choose to drink two ounces of sugarless colloidal minerals. If need be, add the minerals to a small amount of low sugar juice.

Start the machine at 1 amp. Increase ¼ amp every five minutes until you reach a maximum of 2 amps.

Try to clear your mind and relax, breath deeply. You should be resting throughout the entire session (i.e. not watching television, reading a book or talking on the telephone).

Keep your feet in the foot bath and after the set time is over, the machine will turn itself off.

After the unit stops, remove your feet from the foot bath, and turn the machine off.

Dispose of the water in a proper manner. Note: At the end of a session, the foot bath water will be discolored. Some of this discoloration is due to the minerals in the water or salt you are using and some of it may be the body releasing toxins from your skin in to the water.

### Cleaning the Water Module

After each foot bath, rinse the electrode 1 under running water and scrub with a small brush (do not use a wire brush).

After rinsing, soak the module in a soapy water or diluted Silver Wonder solution in a sealable container (large enough for the module) for 5 minutes.

Silver Wonder Solution: Mix 8 oz. Concentrate in one gallon water of Wal-Mart brand Artesian or Spring Water (micron filtered and ozonated) or non-chlorinated water.

### Water Bio-Electric Section

What to do if the readout is less then one (Water conductivity is too low):

Turn the unit back on, add a pinch of salt to the water around the perimeter of the container. Do not sprinkle salt directly over the water module. Stir the water gently with your hand or feet until the salt dissolves.

Keep adding a pinch of salt until the readout shows 2. If the readout goes above 2, you have added too much salt, pour the water out and start over or turn the power control adjust knob slowly to the right (clockwise) until the readout reaches 2.

What to do if the readout is more than two (Water conductivity is too high):

This may cause the circuit breaker to trip. If your circuit breaker has tripped, your green indicator light will not be on.

For your machine to work under these conditions, you will need to adjust the power to the water module. To do this, turn the power control adjust knob to the right (clockwise) until the readout reaches 2.

### Suggestions for Use on Children

4-7 years old: Foot bath sessions for 10-15 minutes.

8-12 years old: Foot bath sessions for 15-20 minutes.

13-17 years old: Foot bath session for 20-35 minutes.
Always supervise children during their foot bath.

### Troubleshooting

Check to see if the power supply is correctly plugged into the wall.

Check to see if the machine is turned on.

If the green indicator light is not on, the circuit breaker may need to be reset.

If the green indicator light still does not come on, unplug the power supply from the wall and check the one external fuse. If the fuses need replacing, replace with the same rating as marked.

Check to see is the DC cable is connected to the water module and the power supply correctly and firmly.

If the unit is still not working, contact a repair technician.

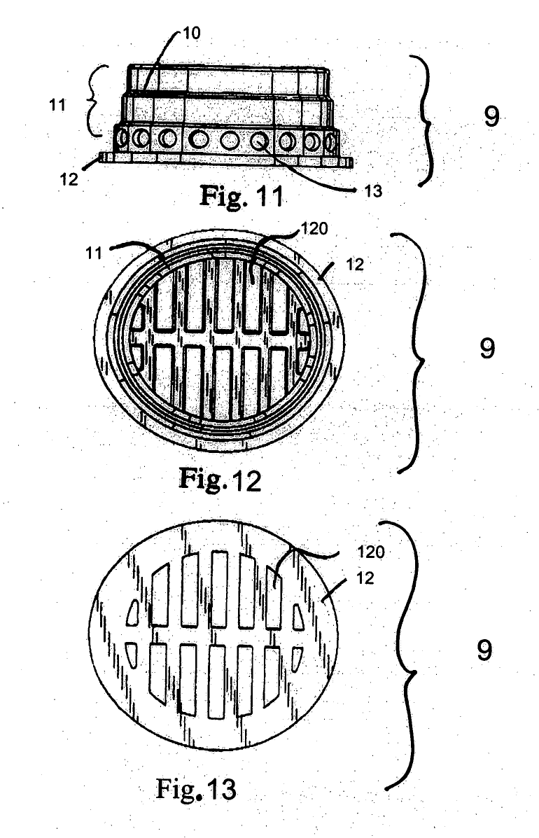

Referring next to FIGS. 11-13 the stand 9 is made of rubber, plastic or nylon and is used to prevent a short circuit in the electrode when in use in a metallic tub. Slots 120 in the bottom 12 and holes 13 allow water to flow freely between the cathode/anode.

FIG. 14 is a schematic diagram of the power source. Anode 3 connected to conductor 5 to series amp meter 80 then through series current limiting device (fuse) 206 to positive terminal of capacitor 204. Positive of indicator lamp 219 is connected to amp meter 80 positive. Cathode 2 connected to conductor 6 to negative terminal of capacitor 204. Alternating current power source 201 connected to isolation transformer 202 through series timer switch 82 and manual dimmer control 81. This forms a variable voltage AC power source to the primary circuit. Secondary of transformer 202 connected to bridge rectifier 203. Positive output of bridge connects to capacitor 204 positive and to bleed resistor 205. Negative output of bridge connects to capacitor 204 negative, indicator lamp 219 and to bleed resistor 205. Typical maximum no-load (open circuit) voltage is approx 35-Volts DC.

### AC/DC Power Source Operation

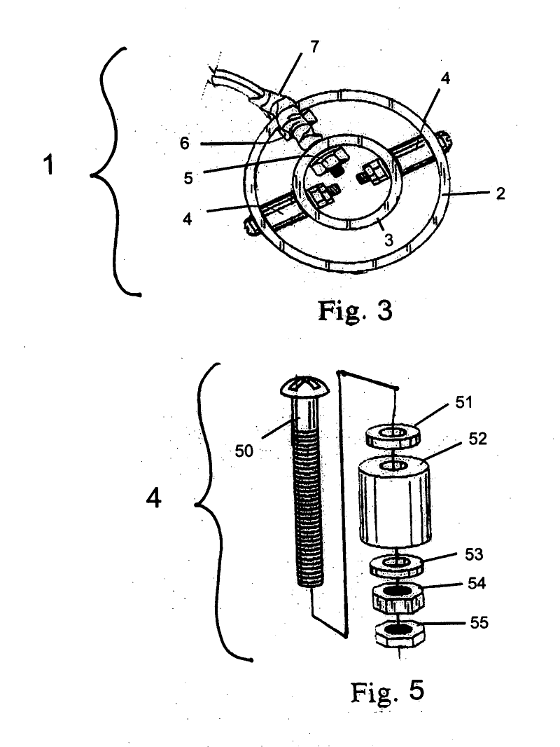

Anode 3 and cathode 2 submerged in an electrolyte 211 with the body member commonly the feet 60 FIG. 6 (not shown). Variable timer switch 82 is closed applying power to primary circuit. Variable AC voltage source 81 rectified and filtered by transformer 202, bridge 203, and capacitor 204. Creating a variable voltage DC (direct current) source, user sets current to desired level shown by amp meter 80. By way of example, current is set to 1-amper with a typical range being 1-4 amperes. This establishes a current flow (via ions) and potential/charge levels in the electrolyte 72. FIG. 3 teaches potential/charge levels as a function of distance 216 as measured by voltmeter 212. User adjusts the current level via electrolyte conductivity and control 81. Typical operation current begins at 1-amper increasing ¼-amper each 5-minutes over a 35-minute period. Treatment period is set with timer 82 values are offered by way of example and not limitation. To support higher currents electrolyte conductivity increased by added more salts to the water/electrolyte 72. An alternate embodiment is an, constant current source substantially equivalent to the simple variable voltage source disclosed.

FIG. 15 is a graph of measured electrolyte/water potential verses distance for two current levels. Electrode probe 215 moved horizontally from top of anode 3 return or zero-potential connected to cathode 2. Potential at zero distance from anode 3 maximum decreasing exponentially with horizontal distance 216 as measured by meter 212. Meter 212 is not part of the invention only shown to teach the electrolyte 72 potential/charge. Curve 301 is electrolyte 72 potential/charge in volts as a function of distance 216 at 1-amper. Curve 303 is electrolyte 72 potential/charge in volts as a function of distance 216 at 3-amperes. Voltage/potential curves follow as predicted by classical electrostatics. The net effect is to raise the water potential a large distance from the anode. Polarities are negative for reversed anode and cathode connections.

Although the present invention has been described with reference to preferred embodiments, numerous modifications and variations can be made and still the result will come within the scope of the invention. No limitation with respect to the specific embodiments disclosed herein is intended or should be inferred. Each apparatus embodiment described herein has numerous equivalents.

1. An electrode comprising:
an outer cylindrical conductor;
an inner cylindrical conductor;
a plurality of members securing the inner cylinder inside the outer cylinder;
a power source connected to the inner and outer cylinders;
a basin having a conductive fluid therein;
wherein the electrode is immersed in the fluid;
a current ranging from 0.1 to 4 amps controllably passing between the conductors; and
wherein the power source has a failsafe circuit breaker preventing a set power limit from passing between the electrodes.

2. The electrode of claim 1, wherein the outer cylinder is about 50.8 mm (2 inches) wide and 76.4 mm (3 inches) high, and the inner cylinder is about 25.4 mm (1 inch) wide and 101.6 mm (4 inches) high.

3. The electrode of claim 2, wherein the plurality of members each further comprises a bolt having an insulating sleeve.

4. The electrode of claim 1 further comprising a stand forming an insulator between the conductors.

5. The electrode of claim 1, wherein the power source further comprises an AC/DC transformer based circuit having a fuse, a circuit breaker and a timer.

6. The electrode of claim 2, wherein a surface area of the outer conductor relative to a surface area of the inner conductor is about 3:1.
